(19) United States
(12) Patent Application Publication
(10) Pub. No.: US 2008/0177301 A1
(43) Pub. Date: Jul. 24, 2008

(54) APPARATUS AND METHOD FOR ANCHORING A PROSTHETIC STRUCTURE TO A BODY TISSUE

(75) Inventor: Lars G. Svensson, Gates Mills, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 11/860,984

(22) Filed: Sep. 25, 2007

Related U.S. Application Data
(60) Provisional application No. 60/848,820, filed on Oct. 2, 2006.

(51) Int. Cl. A61B 17/04

(52) U.S. Cl. 606/228; 606/232

(57) ABSTRACT

A suture member having first and second ends longitudinally separated by a suture midsection is provided. The suture member is fastened to the prosthetic structure with at least one of the first and second ends being located adjacent a tissue-contacting surface of the prosthetic structure. At least one terminal feature is formed on at least one of the first and second ends located adjacent the tissue-contacting surface of the prosthetic structure. The body tissue is engaged with at least one terminal feature to anchor the prosthetic structure to the body tissue. An apparatus for anchoring a prosthetic structure to a body tissue is also provided.

### RELATED APPLICATION

This application claims priority from U.S. Provisional Application No. 60/848,820, filed Oct. 2, 2006, the subject matter of which is incorporated herein by reference.

### TECHNICAL FIELD

The present invention relates to an apparatus and method for anchoring a prosthetic structure to a body tissue and, more particularly, to anchoring a prosthetic structure to a body tissue using a suture member and at least one terminal feature.

### BACKGROUND OF THE INVENTION

In recent years, methods and devices have been developed for inserting a prosthetic structure, discussed herein as being a graft, in a percutaneous or endovascular manner at a desired location within a patient's body. These techniques typically involve a catheter-carried tubular graft delivered through the vascular system to the desired location. The graft may include a tubular graft body supported by an expandable stent, either self-expanding or balloon-expanding. The balloon-expanding type of stent naturally requires an expansion balloon, while the self-expanding type is simply deployed from the end of a tubular sheath.

When the graft installation procedure does not involve cardiopulmonary bypass, blood continues to flow through the vascular system. Certain complications may arise in anchoring the graft to the inner wall of the vessel, because of the blood flow through the vessel both during the procedure and afterward. Indeed, the risk of grafts migrating within a body lumen is a problem in many locations, not just in a blood vessel. Surgeons often require various minimum lengths of healthy body lumen wall adjacent the target location for anchoring the graft and allowing for the graft to shift before an endovascular graft repair is indicated, or else a conventional invasive technique must be used. The latter may cause increased discomfort for the patient or even remove the graft procedure as an option for the patient's treatment. Therefore, it is desirable to anchor the graft endovascularly and with a minimum of healthy body lumen wall adjacent the desired implantation site.

A number of techniques have been proposed for anchoring grafts in a desired location, such as by suturing, stapling, or wiring the graft to the adjacent body tissue. However, puncturing through the body tissue with the suture member, staple, or wire may damage the body tissue and cause complications. Alternatively, some grafts have portions extending beyond the graft body which may be bent outward into contact with the body tissue, either through pre-shaping using a memory alloy or from expansion of a balloon against the extension portion. These extension portions may be complicated and expensive to provide, and may be difficult to custom-fit to a particular application of the graft if adjacent body tissue structures are different than those anticipated by the graft manufacturer.

Alternatively, grafts may be allowed to become naturally fixed to the vessels by tissue ingrowth. For example, some manufacturers have provided grafts having texturized surfaces to provide increased surface area for the body tissue to grow into. One such texturized graft is knitted, and includes a plurality of loops extending from the exterior surface of the graft. One known drawback associated with this configuration is that the knitted graft, which is composed of loosely looped fabrics or threads, is very easily expandable. Such a graft may be subject to undesirable stretching and/or deformation within the body. Another type of texturized graft, an example of which is disclosed in U.S. Pat. No. 6,663,667, is equipped with a plurality of loosely associated threads or fibers, which may be affixed to, woven into, or sewn to the exterior surface of the graft.

Neither of these texturized grafts, however, sufficiently encourages tissue ingrowth into the graft in a suitably timely manner to resist initial migration of the graft. Such ingrowth is a natural bodily process that may be used to anchor the graft, and prevent undesirable migration. Nevertheless, the ingrowth happens gradually over time and will not assist in maintaining the graft in the desired position at or shortly after the time of installation.

Accordingly, it is desirable to provide an apparatus and method for use of an anchor which maintains a prosthetic structure in a desired relationship with a body tissue starting at the time of installation and promoting tissue ingrowth into the prosthetic structure to assist in long-term anchoring of the prosthetic structure to the body tissue.

### SUMMARY OF THE INVENTION

In an embodiment of the present invention, a method of anchoring a prosthetic structure to a body tissue is described. A suture member having first and second ends longitudinally separated by a suture midsection is provided. The suture member is fastened to the prosthetic structure with at least one of the first and second ends being located adjacent a tissue-contacting surface of the prosthetic structure. At least one terminal feature is formed on at least one of the first and second ends located adjacent the tissue-contacting surface of the prosthetic structure. The body tissue is engaged with at least one terminal feature to anchor the prosthetic structure to the body tissue.

In an embodiment of the present invention, an apparatus for anchoring a prosthetic structure to a body tissue is described. The apparatus includes a suture member having first and second ends longitudinally separated by a suture midsection. The suture member is connected to the prosthetic structure. At least one of the first and second ends is located adjacent a tissue-contacting surface of the prosthetic structure. At least one terminal feature is associated with at least one of the first and second ends and is adapted to engage the body tissue for anchoring the prosthetic structure to the body tissue.

### BRIEF DESCRIPTION OF THE DRAWINGS

For a better understanding of the invention, reference may be made to the accompanying drawings, in which:

FIG. 1 is a top view of one embodiment of the present invention in the environment of a prosthetic structure;

FIG. 2 is a top view of the embodiment of FIG. 1 in a first mode;

FIG. 3 is a top view of the embodiment of FIG. 1 in a second mode;

FIG. 4 is a cross-sectional view taken along line 4-4 of FIG. 3; and

FIGS. 5A-5F depict a sequence of manufacture of the embodiment of FIG. 1.

### DESCRIPTION OF EMBODIMENTS

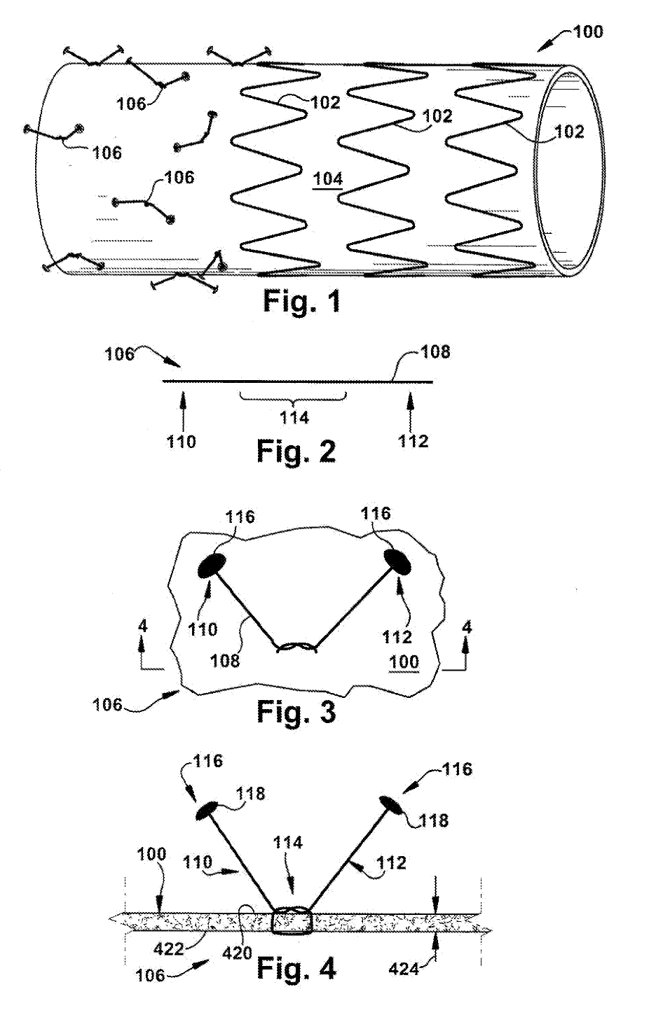

In accordance with the present invention, FIG. 1 depicts a prosthetic structure, discussed herein as being a graft 100, but the present invention may be used with any suitable prosthetic structure. The graft 100 includes one or more self-expanding or balloon-expandable stents 102 and optionally is lined with a graft body 104. At least one apparatus 106 for anchoring the graft 100 to a body tissue (not shown) is provided to the graft 100. The apparatuses 106 are depicted in FIG. 1 as being clustered near one end of the graft 100. However, any desired number of apparatuses 106 may be placed in any suitable arrangement and orientation, and at any location on the graft 100.

FIG. 2 depicts the apparatus 106 of FIG. 1 in a first, uninstalled mode. The apparatus 106 includes a suture member 108 having first and second ends 110 and 112, respectively, longitudinally separated by a suture midsection 114. The suture member 108 may be a wire, monofilament, thread, or the like and may be made of plastic, metal, spun fibers, or any other suitable material.

FIG. 3 depicts the apparatus 106 of FIG. 1 in a second mode in which the suture member 108 is connected to the graft 100. At least one terminal feature 116 is associated with at least one of the first and second ends 110 and 112. As depicted in the Figures, each of the first and second ends 110 and 112 has a terminal feature 116. However, only one of the first and second ends 110 and 112 could have a terminal feature 116, or a single terminal feature could be associated with both of the first and second ends. Regardless of the arrangement or configuration present, the terminal feature 116 is adapted to engage a body tissue (not shown) for anchoring the graft 100 to the body tissue.

The terminal feature 116 may be a separate anchor member attached to at least one of the first and second ends 110 and 112, or may be an integral anchor member formed from at least one of the first and second ends. The term “anchor member” is used to denote any structure which can be used to help maintain the graft 100 in a desired relationship with a body tissue. For example, a hook or barb (not shown) could be formed from, or attached to, at least one of the first and second ends 110 and 112 to serve as an anchor member. The terminal feature 116 may be at least partially insertible into the body tissue for a mechanical engagement therewith. Alternately, the terminal feature 116 could include an adhesive, suction cup, or other noninvasive structure (not shown) to mechanically engage with a surface of the body tissue without being inserted therein.

Whether or not the terminal feature 116 is insertible into the body tissue, the terminal feature may biologically engage with the body tissue. A “biological engagement” is an engagement in which the body tissue exhibits tissue ingrowth over time with at least a portion of the apparatus 106. For example, the body tissue could grow over and subsume at least a portion of the terminal feature 116 and thereby anchor the graft 100.

As shown in the Figures, the anchor member serving as a terminal feature 116 could have a disc shape, with the disc shape being adapted to engage the body tissue. FIG. 4 depicts a cross-sectional view of the disc-shaped terminal feature 116. The disc-shaped terminal feature 116 has a disc rim 118. The disc rim 118 may be dull or sharp. Each disc shape may be flat, concave, convex, or any combination thereof with respect to the corresponding first or second end 110 or 112. A disc-shaped terminal feature 116 may operate much like a mushroom or bell-shaped anchor, commonly used on boats. That is, either a sharp disc rim 118 could dig into the body tissue, or a duller disc rim 118 could catch on an irregular surface of the body tissue, to anchor the graft 100.

As is apparent from the cross-sectional view of FIG. 4, the suture midsection 114 may be connected to the graft 100. More particularly, the graft 100 may have a tissue-contacting surface 420 and an opposing surface 422, with a prosthesis thickness 424 between. The suture midsection 114 may pass through at least a portion of the prosthesis thickness 424 to connect the suture member 108 to the graft 100. In FIG. 4, the suture midsection 114 is shown as passing entirely through the prosthesis thickness 424.

At least one of the first and second ends 110 and 112 may be located adjacent the tissue-contacting surface 420. Moreover, the first and second ends 110 and 112 may be fastened together adjacent the tissue-contacting surface 420. For example, the first and second ends 110 and 112 may be tied together in at least one knot, such as an overhand knot. Alternatively, the first and second ends 110 and 112 may be adhered or fused together or fastened by a separately provided fastener (not shown).

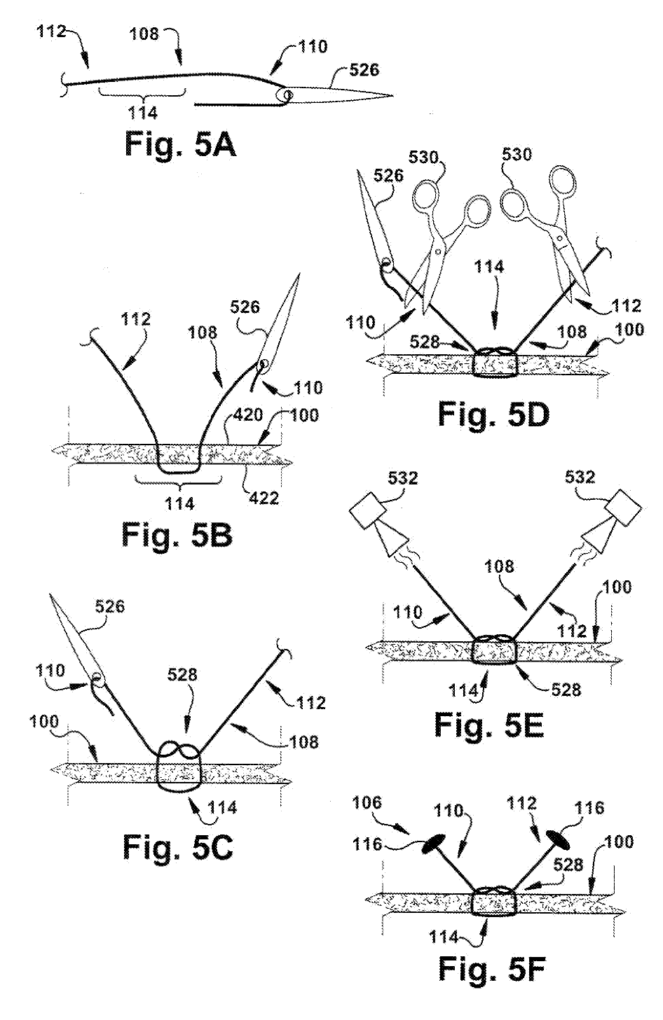

FIGS. 5A-5F depict a sequence of manufacturing steps which may be used to produce the apparatus 106 shown in FIG. 1. First, a length of suture member 108 is provided and a first end 110 of the suture member is connected to a needle 526, as in FIG. 5A. Note that the first end 110 is defined and referenced with respect to the finished apparatus 106, and may be spaced apart from the terminal portion of the initially provided suture member 108 which extends through the needle 526.

Second, the needle 526 is passed through at least a portion of the prosthetic thickness 424 to fasten the suture member 108 to the graft 100. For example, and as shown in FIG. 5B, the needle 526 may be passed completely through the prosthetic thickness 424 from the tissue-contacting surface 420 to the opposing surface 422. The second end 112 is maintained adjacent the tissue-contacting surface 420 and optionally does not pass through the prosthetic thickness 424. The needle 526 is then passed back through the prosthetic thickness 424 of the graft 100 from the opposing surface 422 to the tissue-contacting surface 420 and at least a portion of the suture midsection 114 is pulled through the prosthetic thickness to create the arrangement shown in FIG. 5B.

Next, the first and second ends 110 and 112 may be fastened together. FIG. 5C depicts the tying of an overhand knot 528 to fasten the first and second ends 110 and 112 adjacent the tissue-contacting surface 420. The knots 528, when present, may be of any suitable number and type, and may be drawn as tightly as desired. Alternately, a clip or other fastening device (not shown) could be used to fasten the first and second ends 110 and 112 together.

After the first and second ends 110 and 112 are fastened together, the first end 110 may be disconnected from the needle. As shown schematically in FIG. 5D, scissors 530 or another cutting instrument, such as a scalpel, may be used to sever the suture member 108 in as many locations as needed to trim the first and second ends 110 and 112 into a desired configuration.

Next, at least one terminal feature 116 is formed on or attached to at least one of the first and second ends 110 and 112 located adjacent the tissue-contacting surface 420 of the graft 100. In FIG. 5E, a disc-shaped anchor member is shown as a terminal feature 116 being formed from the first and second ends 110 and 112. More specifically, a heater 532 (shown schematically) supplies heat sufficient to melt a plastic material forming the first and second ends 110 and 112. When the first and second ends 110 and 112 are suitably softened, the plastic is optionally manipulated, such as by being pressed either into a die (not shown) or simply against a flat surface, to form the disc shape of the terminal feature 116. The heater 532 could be combined with a die into a single shaping tool (not shown). When the disc-shaped terminal features 116 have cooled and hardened as desired, the apparatus 106 is in a finished condition and is ready for use, as shown in FIG. 5F.

The above or a similar sequence of steps may be performed as many times as desired to provide a graft 100 with at least one apparatus 106. The apparatuses 106 may be provided to the graft 100 in advance or may be created by the user just before the graft 100 is implanted into the patient. Once the desired number and arrangement of apparatuses 106 have been provided, the graft 100 is placed in the desired location within the patient's body.

As the graft 100 is arranged by the user at the implantation site, at least one terminal feature 116 engages the body tissue adjacent the graft within the patient's body. The terminal feature 116 serves to anchor the graft 100 to the body tissue in a desired manner, initially through a mechanical engagement and, as time progresses, through biological engagement caused by tissue ingrowth. Optionally, some portion of the graft 100 and/or apparatus 106 may include a therapeutic agent or be made from a therapeutic material to encourage such biological engagement.

While aspects of the present invention have been particularly shown and described with reference to the preferred embodiment above, it will be understood by those of ordinary skill in the art that various additional embodiments may be contemplated without departing from the spirit and scope of the present invention. For example, the prosthetic structure 100 could be shaped as a tube, a two-sided sheet, a closed geometric shape, an open frame, or have any other suitable prosthetic structure shape. The body tissue could be any tissue inside or outside the body, including but not limited to an inner or outer surface of blood vessels, skin, muscle, bone, glands, other organs, and the like. The apparatus 106 could be attached to one or more of a stent 102, a graft body 104, an apparatus support (not shown) attached to the prosthetic structure 100, or any other suitable structure. The terminal feature 116 may be produced without heat, depending upon the material of the suture member 108. As with all structures discussed above, the suture member 108 may be made of any suitable material and have any suitable dimensions. One of the first and second ends 110 and 112 could be fastened to the graft 100, with the suture midsection 114 and the other of the first and second ends extending away from the tissue-contacting surface 420. The manufacturing steps depicted in FIGS. 5A-5F could be performed in any suitable order. The apparatuses 106 could be provided in a directional manner to prevent migration of the graft 100 in a predetermined direction with respect to the body tissue. A device or method incorporating any of these features should be understood to fall under the scope of the present invention as determined based upon the claims below and any equivalents thereof.

The method and apparatus of certain embodiments of the present invention, when compared with other apparatus and methods, may have the advantages of maintaining a prosthetic structure in a desired relationship with a body tissue starting at the time of installation and promoting tissue ingrowth into the prosthetic structure to assist in long-term anchoring of the prosthetic structure to the body tissue. Such advantages are particularly worthy of incorporating into the design, manufacture, and operation of prosthetic structures. In addition, the present invention may provide other advantages which have not yet been discovered.

Other aspects, objects, and advantages of the present invention can be obtained from a study of the drawings, the disclosure, and the appended claims.

Having described the invention, I claim:

1. A method of anchoring a prosthetic structure to a body tissue, the method comprising the steps of:
providing a suture member having first and second ends longitudinally separated by a suture midsection;
fastening the suture member to the prosthetic structure with at least one of the first and second ends being located adjacent a tissue-contacting surface of the prosthetic structure;
forming at least one terminal feature on at least one of the first and second ends located adjacent the tissue-contacting surface of the prosthetic structure; and
engaging the body tissue with at least one terminal feature to anchor the prosthetic structure to the body tissue.

2. The method of claim 1, wherein the step of fastening the suture member to the prosthetic structure with at least one of the first and second ends being located adjacent a tissue-contacting surface of the prosthetic structure includes the step of fastening the suture midsection to the prosthetic structure with the first and second ends being located adjacent the tissue-contacting surface of the prosthetic structure.

3. The method of claim 2, wherein the step of fastening the suture midsection to the prosthetic structure with the first and second ends being located adjacent a tissue-contacting surface of the prosthetic structure includes the steps of:
connecting the first end to a needle;
passing the needle through a prosthesis thickness of the prosthetic structure from the tissue-contacting surface to an opposing surface of the prosthetic structure;
maintaining the second end adjacent the tissue-contacting surface;
passing the needle back through the prosthesis thickness of the prosthetic structure from the opposing surface to the tissue-contacting surface;
pulling at least a portion of the suture midsection through the prosthetic thickness;
fastening the first and second ends together adjacent the tissue-contacting surface; and
disconnecting the first end from the needle.

4. The method of claim 3, wherein the step of fastening the first and second ends together adjacent the tissue-contacting surface includes the step of tying the first and second ends together with at least one overhand knot.

5. The method of claim 1, wherein the step of forming at least one terminal feature on at least one of the first and second ends includes the step of forming at least one of the first and second ends into an anchor member.

6. The method of claim 1, wherein the step of forming at least one terminal feature on at least one of the first and second ends includes the step of attaching an anchor member to at least one of the first and second ends.

7. The method of claim 1, wherein the terminal feature has a disc shape, and the step of engaging the body tissue with at least one terminal feature includes the step of contacting the body tissue with the disc shape.

8. The method of claim 7, wherein the step of engaging the body tissue with at least one terminal feature includes at least partially embedding the terminal feature into the body tissue.

9. The method of claim 1, wherein the terminal feature is at least one of a barb and a hook, and the step of engaging the body tissue with at least one terminal feature includes at least partially inserting the terminal feature into the body tissue.

10. The method of claim 1, wherein the step of engaging the body tissue with at least one terminal feature includes permitting the body tissue to biologically engage with the terminal feature.

11. An apparatus for anchoring a prosthetic structure to a body tissue, the apparatus comprising:
a suture member having first and second ends longitudinally separated by a suture midsection;
the suture member being connected to the prosthetic structure;
at least one of the first and second ends being located adjacent a tissue-contacting surface of the prosthetic structure; and
at least one terminal feature associated with at least one of the first and second ends and adapted to engage the body tissue for anchoring the prosthetic structure to the body tissue.

12. The apparatus of claim 11, wherein the suture midsection is connected to the prosthetic structure.

13. The apparatus of claim 11, wherein the tissue-contacting surface is separated from an opposing surface of the prosthetic structure by a prosthesis thickness, the suture midsection passing through at least a portion of the prosthesis thickness to connect the suture member to the prosthetic structure.

14. The apparatus of claim 12, wherein the first and second ends are fastened together adjacent the tissue-contacting surface.

15. The apparatus of claim 11, wherein the terminal feature is an anchor member formed from at least one of the first and second ends.

16. The apparatus of claim 11, wherein the terminal feature is an anchor member attached to at least one of the first and second ends.

17. The apparatus of claim 11, wherein the terminal feature has a disc shape, the disc shape being adapted to engage the body tissue.

18. The apparatus of claim 11, wherein the terminal feature is at least partially insertible into the body tissue.

19. The apparatus of claim 18, wherein the terminal feature is at least one of a barb and a hook.

20. The apparatus of claim 11, wherein the terminal feature biologically engages with the body tissue.
